(19) United States
(12) Patent Application Publication
(10) Pub. No.: US 2013/0202539 A1
(43) Pub. Date: Aug. 8, 2013

(54) ORAL CARE COMPOSITIONS

(71) Applicants: Eleanor Margaret D'Agostino, Sharnbrook (GB); Nicholas David Hedges, Sharnbrook (GB); Asish Nandi, Sharnbrook (GB); Jordan Todorov Petkov, Bebington (GB)

(72) Inventors: Eleanor Margaret D'Agostino, Sharnbrook (GB); Nicholas David Hedges, Sharnbrook (GB); Asish Nandi, Sharnbrook (GB); Jordan Todorov Petkov, Bebington (GB)

(21) Appl. No.: 13/814,748

(22) PCT Filed: Jul. 21, 2011
(86) PCT No.: PCT/EP11/62532

§ 371 Date: Apr. 18, 2013

(30) Foreign Application Priority Data
Aug. 12, 2010 (EP) 10172686.7

(51) Int. Cl. A61K 8/64; A61Q 11/00

(52) U.S. Cl. CPC A61K 8/64; A61Q 11/00
USPC 424/49

(57) ABSTRACT

The invention provides an oral care composition obtainable by: (i) preparing an oil-in-water emulsion by dispersing an oil phase into an aqueous continuous phase, the aqueous continuous phase comprising an oil-in-water emulsifier which is selected from one or more hydrophobins, so that emulsified particles of oil phase are formed which are emulsified with the one or more hydrophobins; and (ii) combining the emulsion so obtained with an oral care base formulation which is suitable for treating the surfaces of the oral cavity. Compositions of the invention demonstrate enhanced deposition of oil phase components onto oral cavity surfaces such as tooth enamel and tooth dentine, and in particular onto damaged surfaces such as scratched and demineralised enamel.

### FIELD OF THE INVENTION

The present invention relates to oral care compositions which provide enhanced delivery of oil phase components to oral cavity surfaces, such as in particular the tooth enamel and dentinal surfaces.

### BACKGROUND OF THE INVENTION

Active ingredients are commonly utilized in oral care products to provide therapeutic benefits (such as the treatment of caries, tooth sensitivity, tooth erosion or gingivitis), or to provide cosmetic benefits within the oral cavity (such as increased tooth whiteness or reduced oral malodour).

Delivery of active ingredients to a site of action within the oral cavity such as a tooth enamel or dentinal surface may be a requirement for obtaining an efficacious response.

Another important factor may include the exposure or contact time of an active ingredient to be treated, in particular if slow or extended release of an active ingredient is desired. If an active ingredient does not retain contact with a surface for a sufficiently long period of time, then efficacy may not be maximized or even achieved at all.

The present inventors have found that hydrophobins can be used to enhance delivery of oil phase components from oral care compositions to oral cavity surfaces.

Compositions of the invention demonstrate enhanced deposition of oil phase components onto oral cavity surfaces such as tooth enamel and tooth dentine, and in particular onto damaged surfaces such as scratched and demineralised enamel.

Furthermore the deposited material appears to be retained well on the treated surface, even in the presence of surface active agents and also after brushing or rinsing.

U.S. Pat. No. 6,117,415 describes a toothpaste containing a bioadhesive submicron oil-in-water emulsion for prolonged local delivery of antibacterial compounds such as chlorhexidine. The antibacterial is entrapped in the oil phase. However, in that system, the oil phase is first emulsified to submicron size using a nonionic surfactant emulsifier (such as polyoxyethylene sorbitan ester). In a further stage the submicron oil particles are coated with a mucoadhesive polymer (such as hydroxypropylmethylcellulose). The coating of mucoadhesive polymer is required in order to effect delivery to the mucous surfaces of the mouth and prolonged release of the antibacterial.

### SUMMARY OF THE INVENTION

The present invention provides an oral care composition obtainable by:

(i) preparing an oil-in-water emulsion by dispersing an oil phase into an aqueous continuous phase, the aqueous continuous phase comprising an oil-in-water emulsifier which is selected from one or more hydrophobins, so that emulsified particles of oil phase are formed which are emulsified with the one or more hydrophobins; and
(ii) combining the emulsion so obtained with an oral care base formulation which is suitable for treating the surfaces of the oral cavity.

The invention also provides a method of enhancing delivery of oil phase components from oral care compositions to oral cavity surfaces, the method comprising treating the surfaces with the oral care composition described above.

In another aspect the invention provides the use of one or more hydrophobins for enhancing delivery of oil phase components from oral care compositions to oral cavity surfaces.

### DETAILED DESCRIPTION OF THE INVENTION

A first stage of the preparation process used to make the oral care composition of the invention involves preparing an oil-in-water emulsion by dispersing an oil phase into an aqueous continuous phase.

### Dispersed Oil Phase

The oil phase may generally be formed from any physiologically acceptable lipophilic material having a liquid or semi-solid consistency at 25° C.

Lipophilic materials suitable for use as oil phase components in the invention include both natural and synthetically produced oils.

Specific examples of suitable oil phase components include naturally or synthetically derived liquid hydrocarbons such as liquid paraffin, squalane, squalene and mineral oil; fatty esters having 6 to 50 carbon atoms in a molecule such as glyceryl monooleate, glyceryl monolinoleate, glyceryl monoisostearate, cetyl isooctanoate, octyldodecyl myristate, isopropyl myristate, isopropyl palmitate, isocetyl stearate, octyldodecyl oleate, sorbitan monooleate, sorbitan monopalmitate, sucrose mono-, di- or tri-palmitate, glyceryl trioctanoate and glyceryl triisostearate; higher fatty acids having 6 to 50 carbon atoms in a molecule such as isostearic acid, oleic acid, hexanoic acid and heptanoic acid; aliphatic higher alcohols having 6 to 50 carbon atoms in a molecule, such as isostearyl alcohol and oleyl alcohol; cyclic or linear organopolysiloxanes such as octamethyl cyclotetrasiloxane, decamethyl cyclopentasiloxane, dimethylpolysiloxane oils and methylphenylpolysiloxane oils; triglyceride oils derived from plant sources such as castor oil, sunflower oil, olive oil, jojoba oil, rapeseed oil, soybean oil, palm kernel oil, babassu kernel oil and coconut oil; and medium-chain triglyceride (MCT) oils, which may generally be defined as mixtures of medium chain saturated fatty acids ranging from caproic to lauric (C₆ to C₁₂), in their triglyceride form, and are typically obtainable from the fractionation of coconut oil.

Mixtures of any of the above described materials may also be used, and may be preferred in some cases. For example liquid materials may be used as diluents or carriers for semi-solid materials in order to improve processability.

Specific examples of useful liquid materials include the MCT oils as defined above.

Specific examples of useful semi-solid materials include long chain fatty acid (C₁₂ to C₂₂) monoglycerides, in particular long chain unsaturated fatty acid (C₁₂ to C₂₂) monoglycerides such as glyceryl monooleate, glyceryl monolinoleate and glyceryl monoisostearate. These materials are able to provide an antisensitivity benefit to teeth. Glyceryl monooleate is particularly preferred.

A preferred oil phase comprises (i) medium-chain triglyceride (MCT) oil (as defined above) and (ii) one or more long chain unsaturated fatty acid (C₁₂ to C₂₂) monoglycerides.

The weight ratio of (i):(ii) in such an oil phase suitably ranges from 10:1 to 1:1, preferably from 8:1 to 2:1.

The oil phase may also include further oral care benefit agents dissolved, dispersed or entrapped therein.

The term “oral care benefit agent” in the context of the present invention generally means any material capable of providing a cosmetic or therapeutic benefit to any of the surfaces found in the oral cavity.

Examples of oral care benefit agents include biologically active substances (such as antioxidants and vitamins), freshening agents for the oral cavity (such as essential oils or their synthetic equivalents), tooth surface whitening agents (such as oil-soluble or oil-dispersible dyes, pigments or pearlescent particles), antimicrobial agents, anticaries agents, tooth remineralising agents and tooth antisensitivity agents.

Preferred oral care benefit agents are those materials capable of providing a cosmetic or therapeutic benefit to the tooth enamel and/or the tooth dentinal surfaces.

### Aqueous Continuous Phase

The aqueous continuous phase (into which the oil phase is dispersed) generally comprises at least 10%, preferably at least 20% by weight water based on the total weight of the aqueous continuous phase.

The aqueous continuous phase may if necessary include a thickener in order to reduce creaming or coalescence of the particles of the dispersed oil phase. Examples of suitable thickeners include organic polyols having 3 or more hydroxyl groups in the molecule (hereinafter termed “organic polyols”). Examples of such materials include glycerol, sorbitol, xylitol, mannitol, lactitol, maltitol, erythritol, and hydrogenated partially hydrolyzed polysaccharides. The most preferred organic polyol is sorbitol. Mixtures of any of the above described materials may also be used.

The total amount of thickener in the aqueous continuous phase will depend on the particular type chosen, but generally ranges from about 0.1 to 75% by weight based on the total weight of the aqueous continuous phase. When the thickener is one or more organic polyols (as described above), the amount of organic polyol suitably ranges from 35 to 75%, more preferably from 45 to 70% by total weight organic polyol based on the total weight of the aqueous continuous phase.

The aqueous continuous phase comprises an oil-in-water emulsifier which is selected from one or more hydrophobins.

Hydrophobins are a well-defined class of proteins (Wessels, 1997, Adv. Microb. Physio. 38: 1-45; Wosten, 2001, Annu Rev. Microbiol. 55: 625-646) capable of self-assembly at a hydrophobic/hydrophilic interface, and having a conserved sequence:

(SEQ ID No. 1)
Xₙ-C-X₅₋₉-C-C-X₁₁₋₃₉-C-X₈₋₂₃-C-X₅₋₉-C-C-X₆₋₁₈-C-Xₘ

where X represents any amino acid, and n and m independently represent an integer. Typically, a hydrophobin has a length of up to 125 amino acids. The cysteine residues (C) in the conserved sequence are part of disulphide bridges. In the context of this invention, the term hydrophobin has a wider meaning to include functionally equivalent proteins still displaying the characteristic of self-assembly at a hydrophobic-hydrophilic interface resulting in a protein film, such as proteins comprising the sequence:

(SEQ ID No. 2)
Xₙ-C-X₁₋₅₀-C-X₀₋₅-C-X₁₋₁₀₀-C-X₁₋₁₀₀-C-X₁₋₅₀-C-X₀₋₅-
C-X₁₋₅₀-C-Xₘ

or parts thereof still displaying the characteristic of self-assembly at a hydrophobic-hydrophilic interface resulting in a protein film. In accordance with the definition of this invention, self-assembly can be detected by adsorbing the protein to Teflon and using Circular Dichroism to establish the presence of a secondary structure (in general, α-helix) (De Vocht et al., 1998, Biophys. J. 74: 2059-68).

The formation of a film can be established by incubating a Teflon sheet in the protein solution followed by at least three washes with water or buffer (Wosten et al., 1994, Embo. J. 13: 5848-54). The protein film can be visualised by any suitable method, such as labelling with a fluorescent marker or by the use of fluorescent antibodies, as is well established in the art. m and n typically have values ranging from 0 to 2000, but more usually m and n in total are less than 100 or 200. The definition of hydrophobin in the context of this invention includes fusion proteins of a hydrophobin and another polypeptide as well as conjugates of hydrophobin and other molecules such as polysaccharides.

Hydrophobins identified to date are generally classed as either class I or class II. Both types have been identified in fungi as secreted proteins that self-assemble at hydrophobic-hydrophilic interfaces into amphipathic films.

Hydrophobin-like proteins have also been identified in filamentous bacteria, such as Actinomycete and Streptomyces sp. (WO01/74864; Talbot, 2003, Curr. Biol, 13: R696-R698). These bacterial proteins by contrast to fungal hydrophobins, may form only up to one disulphide bridge since they may have only two cysteine residues. Such proteins are an example of functional equivalents to hydrophobins having the consensus sequences shown in SEQ ID Nos. 1 and 2, and are within the scope of this invention.

The hydrophobins can be obtained by extraction from native sources, such as filamentous fungi, by any suitable process. For example, hydrophobins can be obtained by culturing filamentous fungi that secrete the hydrophobin into the growth medium or by extraction from fungal mycelia with 60% ethanol. It is particularly preferred to isolate hydrophobins from host organisms that naturally secrete hydrophobins. Preferred hosts are hyphomycetes (e.g. Trichoderma), basidiomycetes and ascomycetes. Particularly preferred hosts are food grade organisms, such as Cryphonectria parasitica which secretes a hydrophobin termed cryparin (MacCabe and Van Alfen, 1999, App. Environ. Microbiol. 65: 5431-5435).

Alternatively, hydrophobins can be obtained by the use of recombinant technology. For example host cells, typically micro-organisms, may be modified to express hydrophobins and the hydrophobins can then be isolated and used in accordance with the present invention. Techniques for introducing nucleic acid constructs encoding hydrophobins into host cells are well known in the art. More than 34 genes coding for hydrophobins have been cloned, from over 16 fungal species (see for example WO96/41882 which gives the sequence of hydrophobins identified in Agaricus bisporus; and Wosten, 2001, Annu. Rev. Microbiol. 55: 625-646). Recombinant technology can also be used to modify hydrophobin sequences or synthesise novel hydrophobins having desired/improved properties.

Typically, an appropriate host cell or organism is transformed by a nucleic acid construct that encodes the desired hydrophobin. The nucleotide sequence coding for the polypeptide can be inserted into a suitable expression vector encoding the necessary elements for transcription and translation and in such a manner that they will be expressed under appropriate conditions (e.g. in proper orientation and correct reading frame and with appropriate targeting and expression sequences).

The methods required to construct these expression vectors are well known to those skilled in the art.

A number of expression systems may be used to express the polypeptide coding sequence. These include, but are not limited to, bacteria, fungi (including yeast), insect cell systems, plant cell culture systems and plants all transformed with the appropriate expression vectors. Preferred hosts are those that are considered food grade—‘generally regarded as safe’ (GRAS).

Suitable fungal species, include yeasts such as (but not limited to) those of the genera Saccharomyces, Kluyveromyces, Pichia, Hansenula, Candida, Schizo saccharomyces and the like, and filamentous species such as (but not limited to) those of the genera Aspergillus, Trichoderma, Mucor, Neurospora, Fusarium and the like.

The sequences encoding the hydrophobins are preferably at least 80% identical at the amino acid level to a hydrophobin identified in nature, more preferably at least 95% or 100% identical. However, persons skilled in the art may make conservative substitutions or other amino acid changes that do not reduce the biological activity of the hydrophobin. For the purpose of the invention these hydrophobins possessing this high level of identity to a hydrophobin that naturally occurs are also embraced within the term “hydrophobins”.

Hydrophobins can be purified from culture media or cellular extracts by, for example, the procedure described in WO01/57076 which involves adsorbing the hydrophobin present in a hydrophobin-containing solution to surface and then contacting the surface with a surfactant, such as Tween 20, to elute the hydrophobin from the surface. See also Collen et al., 2002, Biochim Biophys Acta. 1569: 139-50; Calonje et al., 2002, Can. J. Microbiol. 48: 1030-4; Askolin et al., 2001, Appl Microbiol Biotechnol. 57: 124-30; and De Vries et al., 1999, Eur J. Biochem. 262: 377-85.

Typically, the hydrophobin is in an isolated form, typically at least partially purified, such as at least 10% pure, based on weight of solids. By “isolated form”, we mean that the hydrophobin is not added as part of a naturally-occurring organism, such as a mushroom, which naturally expresses hydrophobins. Instead, the hydrophobin will typically either have been extracted from a naturally-occurring source or obtained by recombinant expression in a host organism.

Hydrophobin proteins can be divided into two classes: Class I, which are largely insoluble in water, and Class II, which are readily soluble in water.

Preferably, the hydrophobins chosen are Class II hydrophobins. More preferably the hydrophobins used are Class II hydrophobins such as HFBI, HFBII, HFBIII, or Cerato ulmin.

The hydrophobin can be from a single source or a plurality of sources e.g. a mixture of two or more different hydrophobins.

The total amount of hydrophobin in the aqueous continuous phase will generally be at least 0.001%, more preferably at least 0.005 or 0.01%, and generally no greater than 2% by total weight hydrophobin based on the total weight of the aqueous continuous phase.

In order to optimise delivery of the oil phase components, it is particularly preferred that the aqueous continuous phase (into which the oil phase is dispersed) is substantially free of anionic surfactant. The term “substantially free” in this particular context generally means that the aqueous continuous phase comprises less than 1%, more preferably less than 0.1%, most preferably less than 0.01% by total weight anionic surfactant based on the total weight of the aqueous continuous phase.

Examples of anionic surfactants include the sodium, magnesium, ammonium or ethanolamine salts of C₈ to C₁₈ alkyl sulphates (for example sodium dodecyl sulphate), C₈ to C₁₈ alkyl sulphosuccinates (for example dioctyl sodium sulphosuccinate), C₈ to C₁₈ alkyl sulphoacetates (such as sodium dodecyl sulphoacetate), C₈ to C₁₈ alkyl sarcosinates (such as sodium dodecyl sarcosinate), C₈ to C₁₈ alkyl phosphates (which can optionally comprise up to 10 ethylene oxide and/or propylene oxide units) and sulphated monoglycerides.

Anionic surfactants can however be added at a later stage of the preparation process used to make the oral care composition, for example as a component of the oral care base formulations described below.

A typical process used to form the oil-in-water emulsion described above comprises the following steps:

mixing one or more hydrophobins with water and optionally a thickener such as sorbitol to form an aqueous phase;
mixing one or more oil phase components (as described above) in a separate vessel to form an oil phase;
adding the oil phase to the aqueous phase, agitating to form a mixture and subjecting the resultant mixture to a mechanical emulsification treatment, thereby forming an oil-in-water emulsion in which the emulsified particles of oil phase are emulsified with the one or more hydrophobins.

The mechanical emulsification treatment may suitably be carried out using high shear mixing or homogenizing equipment known to those skilled in the art, such as a Silverson® mixer or a Microfluidizer®.

Heating may be employed if necessary to aid processing during any or all of the process steps described above.

A particularly preferred oil-in-water emulsion used to prepare the oral care composition of the invention comprises the following ingredients:

| | |
|---|---|
| | wt % (by weight based on the total |
| | weight of the oil-in-water |
| Ingredient | emulsion) |

Aqueous Continuous Phase

| | |
|---|---|
| Water | from 10 to 60%, preferably from 20 |
| | to 50% |
| Organic polyol | from 40 to 80%, preferably from 50 |
| (e.g. sorbitol) | to 70% |
| Hydrophobin | from 0.01 to 0.5%, preferably from |
| | 0.05 to 0.25% |

Dispersed Oil Phase

| | |
|---|---|
| Long chain unsaturated | from 1 to 10%, preferably from 2 to |
| fatty acid (C₁₂ to C₂₂) | 8% |
| monoglyceride | |
| Medium-chain triglyceride | from 0.2 to 2%, preferably from 0.5 |
| (MCT) oil | to 1.5% |

### Oral Care Base Formulations

A second stage of the process used to prepare the oral care composition of the invention involves combining the oil-in-water emulsion described above with an oral care base formulation which is suitable for treating the surfaces of the oral cavity.

Suitable oral care base formulations may take various product forms. Examples of suitable product forms include dentifrice, mouthwash, tooth powder, chewing gum, lozenge, mouth spray, floss or dental strip.

The amount of oil-in-water emulsion in the final oral care composition will depend on the oral care base formulation used, but generally ranges from 5 to 95% by total weight of the oil-in-water emulsion based on the total weight of the composition.

Preferred oral care base formulations are those which are suitable for brushing and/or rinsing the surfaces of the oral cavity.

Such formulations generally comprise a continuous phase comprising water or monohydric or polyhydric alcohol or a mixture thereof.

Preferably the continuous phase comprises water or polyhydric alcohol or a mixture thereof.

An example of a preferred type of oral care base formulation in the context of the present invention is a dentifrice. The term “dentifrice” generally denotes formulations which are used to clean the surfaces of the oral cavity. The dentifrice is an oral composition that is not intentionally swallowed for purposes of systemic administration of therapeutic agents, but is applied to the oral cavity, used to treat the oral cavity and then expectorated. Typically the dentifrice is used in conjunction with a cleaning implement such as a toothbrush, usually by applying it to the bristles of the toothbrush and then brushing the accessible surfaces of the oral cavity. Preferably the dentifrice is in the form of a paste or a gel (or a combination thereof).

A dentifrice suitable for use in the invention will usually contain a liquid continuous phase in an amount of from 40 to 99% by weight based on the total weight of the dentifrice. Such a liquid continuous phase will typically comprise a mixture of water and polyhydric alcohol in various relative amounts, with the amount of water generally ranging from 10 to 45% by weight (based on the total weight of the dentifrice) and the amount of polyhydric alcohol generally ranging from 30 to 70% by weight (based on the total weight of the dentifrice). Typical polyhydric alcohols include humectants such as glycerol, sorbitol, polyethylene glycol, polypropylene glycol, propylene glycol, xylitol (and other edible polyhydric alcohols), hydrogenated partially hydrolyzed polysaccharides and mixtures thereof.

A dentifrice suitable for use in the invention will generally contain further ingredients to enhance performance and/or consumer acceptability such as abrasive cleaning agent, binder or thickening agent, and surfactant.

For example, a dentifrice will usually comprise an abrasive cleaning agent in an amount of from 3 to 75% by weight based on the total weight of the dentifrice. Suitable abrasive cleaning agents include silica xerogels, hydrogels and aerogels and precipitated particulate silicas; calcium carbonate, dicalcium phosphate, tricalcium phosphate, calcined alumina, sodium and potassium metaphosphate, sodium and potassium pyrophosphates, sodium trimetaphosphate, sodium hexametaphosphate, particulate hydroxyapatite and mixtures thereof.

Furthermore, the dentifrice will usually contain a binder or thickening agent in an amount of from 0.5 to 10% by weight based on the total weight of the dentifrice. Suitable binders or thickening agents include carboxyvinyl polymers (such as polyacrylic acids cross-linked with polyallyl sucrose or polyallyl pentaerythritol), hydroxyethyl cellulose, hydroxypropyl cellulose, water soluble salts of cellulose ethers (such as sodium carboxymethyl cellulose and sodium carboxymethyl hydroxyethyl cellulose), natural gums (such as carrageenan, gum karaya, guar gum, xanthan gum, gum arabic, and gum tragacanth), finely divided silicas, hectorites, colloidal magnesium aluminium silicates and mixtures thereof.

Furthermore, the dentifrice will usually contain a surfactant in an amount of from 0.2 to 5% by weight based on the total weight of the dentifrice. Suitable surfactants include anionic surfactants, such as the sodium, magnesium, ammonium or ethanolamine salts of C₈ to C₁₈ alkyl sulphates (for example sodium dodecyl sulphate), C₈ to C₁₈ alkyl sulphosuccinates (for example dioctyl sodium sulphosuccinate), C₈ to C₁₈ alkyl sulphoacetates (such as sodium dodecyl sulphoacetate), C₈ to C₁₈ alkyl sarcosinates (such as sodium dodecyl sarcosinate), C₈ to C₁₈ alkyl phosphates (which can optionally comprise up to 10 ethylene oxide and/or propylene oxide units) and sulphated monoglycerides. Other suitable surfactants include nonionic surfactants, such as optionally polyethoxylated fatty acid sorbitan esters, ethoxylated fatty acids, esters of polyethylene glycol, ethoxylates of fatty acid monoglycerides and diglycerides, and ethylene oxide/propylene oxide block polymers. Other suitable surfactants include amphoteric surfactants, such as betaines or sulphobetaines. Mixtures of any of the above described materials may also be used.

In a final oral care composition according to the invention, a dentifrice as described above is generally combined with an oil-in-water emulsion as described above in a (dentifrice):(emulsion) weight ratio ranging from 4:1 to 1:4, preferably from 2:1 to 1:2.

Another example of a preferred type of oral care base formulation in the context of the present invention is a mouthwash. The term “mouthwash” generally denotes liquid formulations which are used to rinse the surfaces of the oral cavity and provide the user with a sensation of oral cleanliness and refreshment. The mouthwash is an oral composition that is not intentionally swallowed for purposes of systemic administration of therapeutic agents, but is applied to the oral cavity, used to treat the oral cavity and then expectorated.

A mouthwash composition suitable for use in the invention will usually contain an aqueous continuous phase. The amount of water generally ranges from 70 to 99% by weight based on the total weight of the mouthwash.

A mouthwash composition suitable for use in the invention may also contain a monohydric alcohol such as ethanol, isopropanol or a mixture thereof. If present, the amount of monohydric alcohol typically ranges from 1 to 25%, preferably from 10 to 20% by weight based on the total weight of the mouthwash.

A mouthwash composition suitable for use in the invention will generally contain further ingredients to enhance performance and/or consumer acceptability, such as the humectants and surfactants mentioned above for dentifrices. The amount of humectant generally ranges from 5 to 20% by weight based on the total weight of the mouthwash and the amount of surfactant generally ranges from 0.1 to 5% by weight based on the total weight of the mouthwash.

In a final oral care composition according to the invention, a mouthwash as described above is generally combined with an oil-in-water emulsion as described above in a (mouthwash):(emulsion) weight ratio ranging from 10:1 to 1:1, preferably from 6:1 to 2:1.

Oral care base formulations such as the dentifrices or mouthwashes described above may also contain further optional ingredients customary in the art such as fluoride ion sources, anticalculus agents, buffers, flavouring agents, sweetening agents, colouring agents, opacifying agents, preservatives, antisensitivity agents, delivery-enhancing polymers (such as polymers based on a copolymer of methyl ether with maleic anhydride) and antimicrobial agents.

The invention is further illustrated with reference to the following, non-limiting Examples.

### EXAMPLES

### Example 1

A composition was prepared having ingredients as shown in the following Table:

| | Example 1 |
|---|---|
| Ingredient | (% w/w) |
| Sorbitol aqueous solution (70% a.i.) | 83.8 |
| Glyceryl monooleate (GMO) | 5.0 |
| Medium-chain triglyceride (MCT) oil | 1.0 |
| Sodium dodecyl sulphate | 0.5 |
| Hydrophobin* | 0.15 |
| Water | q.s. |

The composition was prepared as follows:

The glyceryl monooleate was mixed with the MCT oil and placed in a water bath at 90° C. to from an oil phase;

The sorbitol aqueous solution (70% a.i.) was mixed with the hydrophobin and heated to 90° C. to form an aqueous phase;

The oil phase and the sorbitol phase were each allowed to equilibrate for 10 minutes;

The oil phase was added to the aqueous phase at 90° C.;

The hot mixture was then mixed in a Silverson® mixer at 90° C. for 60 seconds;

The emulsion was cooled by transferring to an ice bath while mixing in the Silverson® mixer for 90 seconds;

The sodium dodecyl sulphate was added in aqueous solution to the emulsion at room temperature and mixed well to form the final composition.

### Evaluation of Composition of Example 1

### Microscopy Studies

The composition of Example 1 was examined under a confocal microscope at various time intervals: freshly prepared, after 4 days and after 3 weeks. The composition appeared stable with no gel aggregates visible even after 3 weeks. The composition was only slightly mobile on the glass slide suggesting deposition of the GMO onto the glass surface.

Deposition onto Tooth Dentine

The deposition of the composition of Example 1 onto dentine (both with and without salivary pellicle) was assessed using 8×8 mm bovine tooth slabs.

The assessment methodology was as follows:

Tooth slabs were sonicated in a beaker of distilled water for 5 minutes using an ultrasonic bath;

To form a pellicle the tooth slabs were placed in saliva for at least 2 hours;

The slabs were then treated with 25 μL of the composition of Example 1 (stained with Nile blue fluorescent dye) and incubated at room temperature for 5 minutes;

The slabs were then dabbed with tissue and deposition was assessed using fluorescent confocal microscopy and stereo macroscopy;

The slabs were then washed under a continuous flow of tap water for 5 seconds and re-examined;

The slabs were washed twice more and deposition examined after each wash;

The slabs were then brushed with a commercial (Close-Up®) toothpaste for about 10 seconds, washed under tap water for 5 seconds and re-examined.

Assessment of the treated slabs showed deposition of the composition of Example 1 onto the slabs. Furthermore the deposit showed good resistance to the washing and brushing treatment carried out.

A comparative test was also carried out using a control formulation. The control formulation was prepared using equivalent ingredients and methodology, except that the oil phase was emulsified with sodium dodecyl sulphate instead of hydrophobin.

Deposition of this control formulation onto the treated slabs was observed to be significantly inferior to that of the composition of Example 1.

### Example 2

A composition was prepared as described above in Example 1. 1 part of this composition was mixed with 4 parts of an oral care base formulation in the form of a mouthwash. The final composition is indicated below.

| | Example 2 |
|---|---|
| Ingredient | (% w/w) |
| Sorbitol aqueous solution (70% a.i.) | 16.76 |
| Glyceryl monooleate (GMO) | 1.0 |
| Medium-chain triglyceride (MCT) oil | 0.2 |
| Sodium dodecyl sulphate | 0.35 |
| Hydrophobin* | 0.03 |
| Water | q.s. |
| Flavour oil | 0.25 |
| Benzyl alcohol | 0.3 |
| Sodium saccharin | 0.07 |
| Phenoxyethanol | 0.3 |
| Sodium fluoride | 0.05 |

### Example 3

A composition was prepared as described above in Example 1. 1 part of this composition was mixed with 1 part of an oral care base formulation in the form of a dentifrice. The final composition is indicated below.

| | Example 3 |
|---|---|
| Ingredient | (% w/w) |
| Sorbitol aqueous solution (70% a.i.) | 45 |
| Sodium saccharin | 0.2 |
| Polyethylene glycol 32M | 2 |
| C.I. 77891 (TiO₂) | 1 |
| Thickening silica | 8 |
| Abrasive silica | 10 |
| Sodium dodecyl sulphate | 1.5 |
| Flavour oil | 1 |
| Sodium carboxymethyl cellulose | 0.9 |
| Sodium fluoride | 0.32 |
| Glyceryl monooleate (GMO) | 2.5 |
| Hydrophobin* | 0.075 |
| Medium-chain triglyceride (MCT) oil | 0.5 |
| Water | q.s. |

[*The specific hydrophobin used was Class II Hydrophobin HFBII, obtained from VTT Biotechnology, Finland. It had been purified from Trichoderma reesei essentially as described in WO00/58342 and Under et al., 2001, Biomacromolecules 2: 511-517.]

1. An oral care composition obtainable by:
(i) preparing an oil-in-water emulsion by dispersing an oil phase into an aqueous continuous phase, the aqueous continuous phase comprising an oil-in-water emulsifier which is selected from one or more hydrophobins, so that emulsified particles of oil phase are formed which are emulsified with the one or more hydrophobins; and
ii) combining the emulsion so obtained with an oral care base formulation which is suitable for treating the surfaces of the oral cavity.

2. An oral care composition according to claim 1, where the hydrophobin is a Class II hydrophobin.

3. An oral care composition according to claim 2, where the Class II hydrophobin is HFBI, HFBII, or a mixture thereof.

4. An oral care composition according to claim 1, in which the oil phase comprises (i) a mixture of medium chain saturated fatty acids ranging from caproic to lauric (C₆ to C₁₂), in their triglyceride form, and (ii) one or more long chain unsaturated fatty acid (C₁₂ to C₂₂) monoglycerides.

5. An oral care composition according to claim 1, in which the aqueous continuous phase (into which the oil phase is dispersed) comprises less than 0.01% anionic surfactant (by total weight anionic surfactant based on the total weight of the aqueous continuous phase).

6. An oral care composition according to any claim 1, in which the oral care base formulation comprises a continuous phase comprising water or polyhydric alcohol or a mixture thereof.

7. An oral care composition according to claim 1, in which the oral care base formulation is suitable for brushing and/or rinsing the surfaces of the oral cavity.

8. An oral care composition according to claim 7, in which the oral care base formulation is a dentifrice or a mouthwash.
